(19) United States
(12) Patent Application Publication
(10) Pub. No.: US 2006/0025779 A1
(43) Pub. Date: Feb. 2, 2006

(54) Tube insertion device and method therefor

(76) Inventor: Janet F. Dutcher, Sioux Falls, SD (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 10/903,625

(22) Filed: Jul. 30, 2004

(51) Int. Cl. A61F 11/00

(52) U.S. Cl. 606/108

(57) ABSTRACT

A tube insertion device that allows for an elongate tube to be retained or clamped within a portion the tube insertion device without substantially increasing an outer diameter of the tube insertion device.

### TECHNICAL FIELD

The present application relates to a tube insertion device, such as a forceps for inserting a tube into a patient.

### TECHNICAL BACKGROUND

Medical situations such as emergencies due to a collapsed lung often require the insertion of elongate tubing within a patient. Such tubing can form a drain to release fluids and/or pressure from the particular area. One example of such a medical situation is the collapse of a lung, and the tubing must be inserted into the chest cavity between the ribs.

The tubing can be inserted in a number of different manners. For example, a distal tip of surgical forceps or a hemostat are disposed between ribs of the patient and used to separate the muscle to create a small opening between the ribs.

When the forceps has formed the required opening, the opening is maintained in an open position by the forceps as the tubing is inserted through the muscle opening into, for example, the chest cavity. However, it can be difficult to manipulate the tubing through the opening due to the presence of the forceps within the opening. Furthermore, this can be further difficult for smaller patients such as children or infants. The size of the combination of the forceps and the tubing further can cause additional trauma to the surrounding tissue and the patient.

What is needed is a way to insert tubing into small openings within patients, including smaller patients, without causing unnecessary trauma to the patient.

### SUMMARY

A tube insertion device such as forceps or a hemostat includes a clamping portion operably coupled with a hinge portion where the clamping portion has opposing clamping members. The clamping members extend from a first clamping member and to a second clamping member distal end, where the clamp members have a first open position and a second closed position. The clamping members further have opposing channels therein, where the channels are recessed away from the gripping surface of the clamping members. The tube insertion device further includes a handle portion that is operably coupled with the hinge portion.

Several options for the tube insertion device are as follows. For example, in one option, the channel is sized and configured to receive tubing therein, such as chest tubing, without substantially collapsing a lumen of the tubing when the clamping members are disposed in the second closed position. In another option, at least a portion of the channels are aligned with longitudinal axis of the clamping members, where optionally the clamping portion or the clamping members are arcuately curved and have a radius. In yet another option, each channel has a U-shaped cross-section, and yet in another option the clamping members include an intermediate portion and the clamping members further have an exit recessed disposed along the intermediate portion.

Several options for the tube insertion device are as follows. For example, along with an exit recess disposed along an intermediate portion of the clamping members, the tube insertion device can further comprise a means for locking the clamping members in a closed position and another option each of the clamping members may have a recess in the recesses of the clamping member oppose each other.

A method is further providing which includes opening and closing clamping members of a tube insertion device, where the clamping members of the tube insertion device have a first outer diameter in a closed position. The method further includes disposing an elongate tubing within a channel of at least one of the clamping members of the tube insertion device, where the tubing has a lumen. The method further includes clamping around a portion of the elongate tubing without substantially collapsing the lumen of the tubing when the clamping members are disposed in the closed position.

Several options for the method are as follows. For instance, in one option, the method further includes locking at least a portion of the tube insertion device when the clamping members are disposed in a closed position, or clamping the portion of an elongate tubing includes clamping the elongate tubing without substantially increasing the first outer diameter when the clamping members are disposed in the closed position. In another option, elongate tubing is disposed adjacent to channel distal end of the clamping member, or is disposed through an exit recess disposed along an intermediate portion of the clamping members. In another option, disposing the elongate tubing within the channel includes disposing elongate tubing within at least two opposing channels of the clamping members.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims and their equivalents.

### BRIEF DESCRIPTION OF THE DRAWINGS

FIG. 1 illustrates a side elevational view of the tube insertion device disposed in a closed configuration as constructed in accordance with at least one embodiment.

FIG. 2 illustrates a side elevational view of the tube insertion device disposed in an open configuration as constructed in accordance with at least one embodiment.

FIG. 3A illustrates a perspective view of a portion of the tube insertion device as constructed in accordance with at least one embodiment.

FIG. 3B illustrates a side elevational view of a clamping member constructed in accordance with at least one embodiment.

FIG. 4 illustrates a cross-sectional view of a clamping member taken along 4-4 of FIG. 3B.

FIG. 5 illustrates a side elevational view of a tube insertion device including an elongate tube disposed therein, as constructed in accordance with at least one embodiment.

FIG. 6 illustrates a second side elevational view of a tube insertion device including an elongate tube disposed therein, as constructed in accordance with at least one embodiment.

### DESCRIPTION OF THE EMBODIMENTS

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

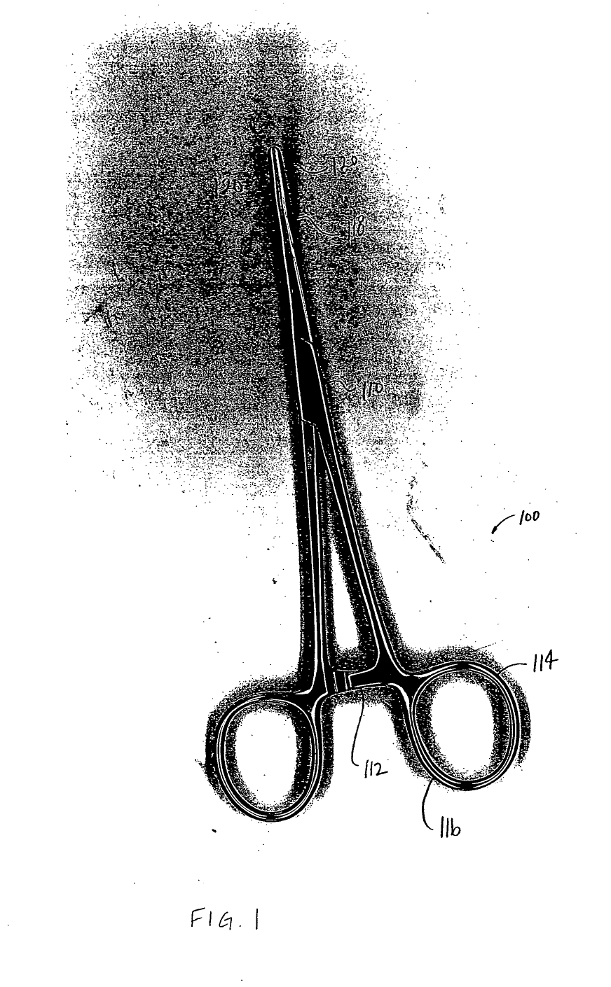

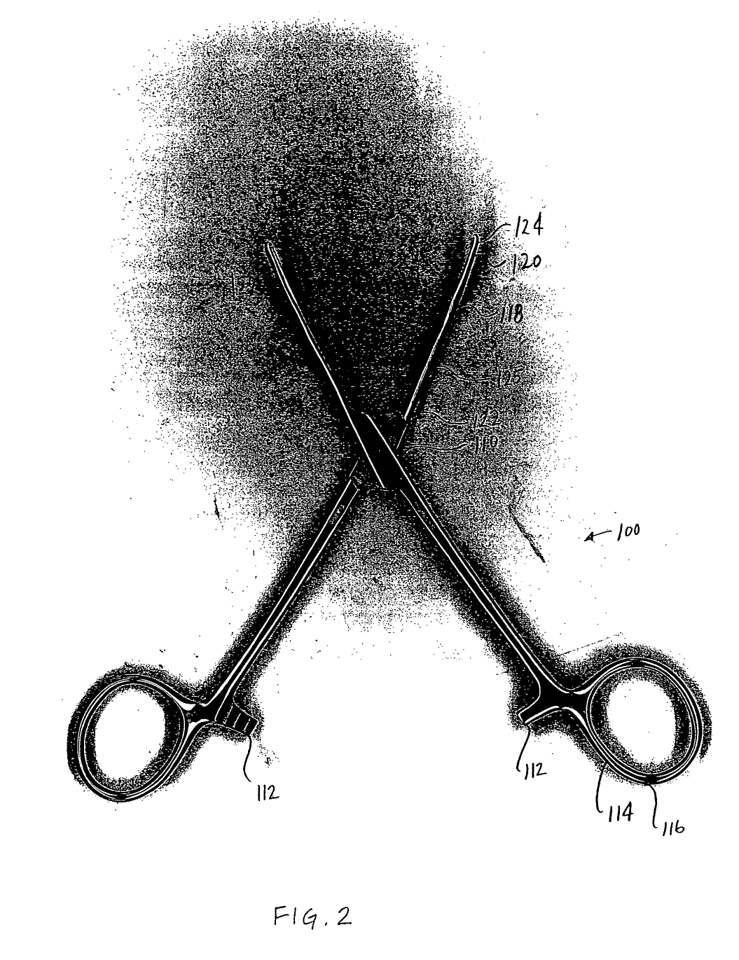

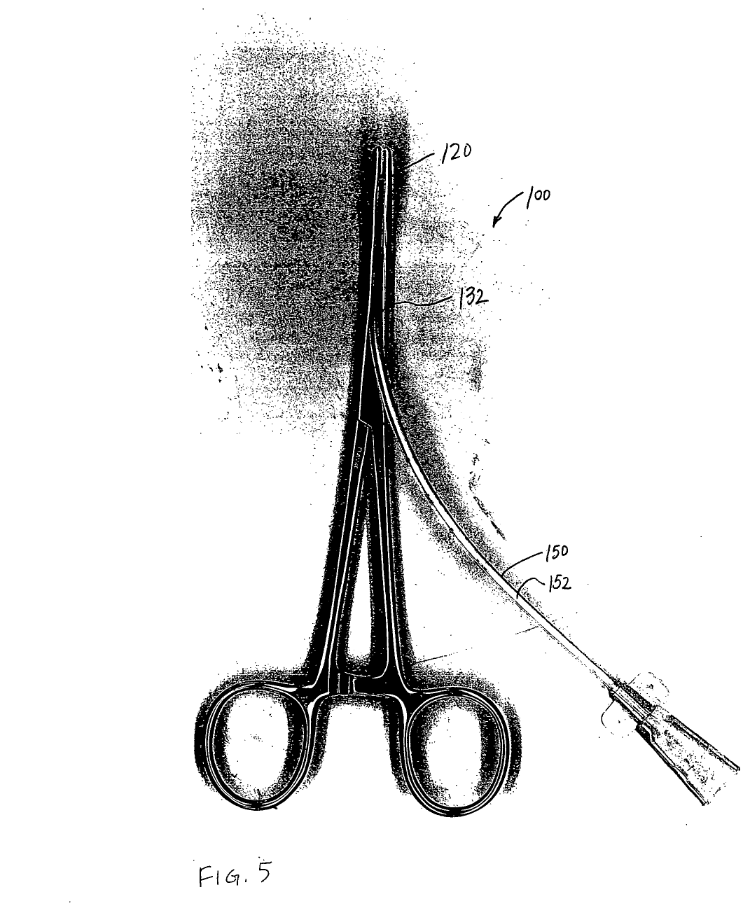

FIGS. 1 and 2 illustrate examples of a tube insertion device 100, where FIG. 1 illustrates the tube insertion device 100 in a closed position, and FIG. 2 illustrates the tube insertion device 100 in an open position. The tube insertion device 100 is generally used to insert tubing, such as elongate tubing (FIG. 5) into a patient. In one example, the tube insertion device 100 includes a pair of forceps. In another option, the tube insertion device 100 includes a hemostat.

The tube insertion device 100 generally includes a clamping portion 118 that is operably connected with a hinge portion 110. A handle portion 116 is further operably connected with the hinge portion 110, and allows for the tube insertion device 100 to be manipulated from the closed position to the open position, and vice versa.

The handle portion 116, in one option, includes finger loops 114 that allow for the users fingers to be inserted therein. It should be noted that other forms of handle portion 116 can be included and are considered within the scope of this document. In a further option, locking features 112 are further included with the tube insertion device 100. In one option, the locking features 112 include a series of interlocking flanges disposed on a respective portions of the handle portion 116 of the tube insertion device 100. In one option, the locking features 112 allow for the tube insertion device 100 to be locked in a closed position, or a clamped position.

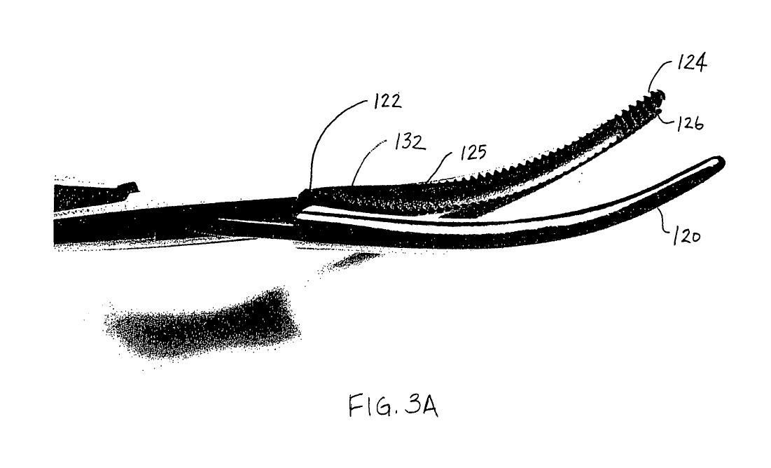

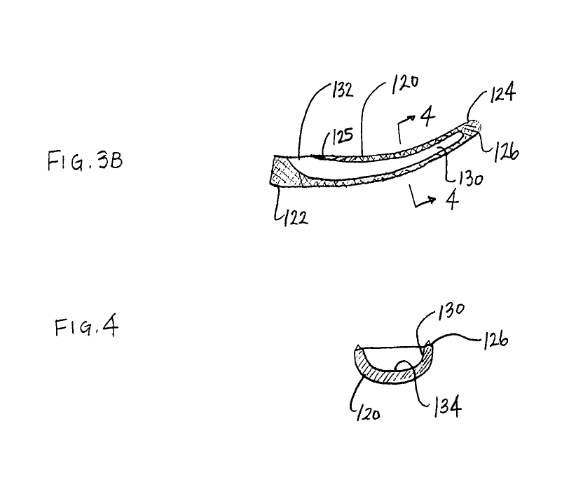

The clamping portion 118 includes opposing clamp members 120, one of which is illustrated in greater detail in FIGS. 3A, 3B, and 4. The clamp members 120 include a first clamp member and a second clamp member where each clamp member 120 includes a first clamp member end 122 extending to a second clamp member distal end 124. Each of the clamp members 120 includes a gripping surface 126, for example, a knurled surface or a surface having one or more projections to allow for items to be grasped by the gripping surface 126.

One or more of the clamp members 120 further includes a channel 130, or alternatively a recess that is substantially aligned with a longitudinal axis of the clamp member 120. In one option, a portion of the channel 130 is substantially aligned with a longitudinal axis of the clamp member 120. The channel 130 is disposed along the clamp member 120 allowing for a device such as elongate tubing to be disposed and retained therein while the tube insertion device 100 (FIG. 1) is disposed in a closed position, thereby allowing for the elongate tubing to be disposed with a patient, as further described below. The channel 130 optionally includes a serrated, knurled, or non-smooth surface.

The channel 130, in one option, is recessed away from the gripping surface 126 of the clamp member 120, and in one option forms a U-shaped channel, as illustrated in FIG. 4. The U-shaped channel is defined in part by an interior surface 134 having an arcuate shape which can receive an arcuate structure, such as elongate tubing, therein. In a further option, the channel 130 extends along the clamp member 120 from near a distal end 124 to an intermediate portion 125 at which an exit recess 132 is disposed. The exit recess 132 allows for the channel 130 to depart from the longitudinal axis of the clamping member 120 and allows for the structure disposed within the channel, such as the elongate tubing, to exit from the clamp member 120 when the tube insertion device 100 is disposed in a closed position. This allows for further options in manipulating the elongate structure. As further illustrated in FIG. 3A, in one option, the clamping members 120 have an arcuate shape with a radius. In yet another option, the clamping members 120 have a distal end that is sharpened to assist in puncturing tissue, for example, between the ribs of a patient.

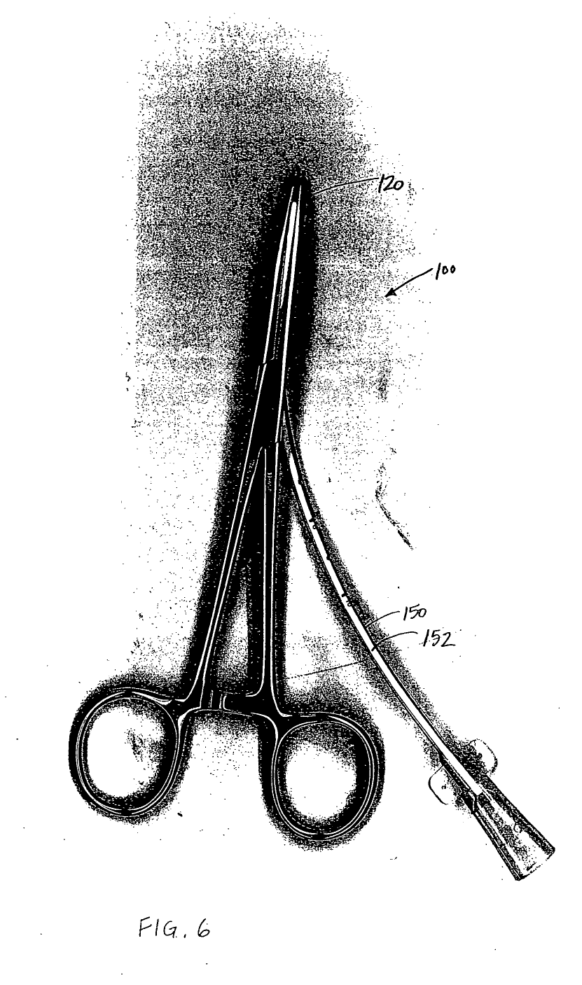

In another option, the tube insertion device 100 further includes elongate tubing 150 disposed within the channel or recess of the device, as illustrated in FIGS. 5 and 6. The tube insertion device 100 allows for the device 100 to be placed in a closed position without substantially collapsing a lumen 152 of the tubing 150, while the device 100 is placed in the closed position. As described above, the recess or channel of the tube insertion device 100 is sized and configured to receive the tubing therein, without substantially collapsing the lumen of the tubing 150 when the clamping members are disposed in the closed position. As illustrated in FIG. 5, the tubing 150 exits from the exit recess 132 of one option of the tube insertion device 100. The exit recess 132 is formed for example, by extending the channel through a side wall of the clamping members, allowing for the lumen of the tubing to remain uncompressed when the tube is inserted into the patient.

During use of the device the clamping members of the tube insertion device are opened and closed, where the clamping members of the tube insertion device have a first outer diameter in a closed position. Elongate tubing as disposed within the channel or recess of at least one of the clamping members of the tube insertion device, where the tubing has a lumen. In one option, a distal end of the tubing is placed adjacent a distal end of the clamping member and/or the channel. The tube insertion device is further used to clamp around a portion of the elongate tubing without substantially collapsing the lumen of the tubing when the clamping members are disposed in the closed position. The elongate tube has optionally disposed through the exit recess disposed along the intermediate portion of the clamping members, as further described above. During clamping of the portion of the elongate tubing, this is done without substantially increasing the first outer diameter of the clamping members when the clamping members are disposed in the closed position.

In a further option, the elongate tubing is disposed with two opposing channels that are disposed within the clamping members and the elongate tubing fits within the channel. It should be noted that any shape or size of the channel in at least one of the clamping members can be used such that it receives the elongate tubing therein. After the device is used to clamp around a portion of the elongate tubing, optionally the device 100 is used to lock in a closed position around the elongate tubing.

Advantageously, a user can use the forceps device to easily manipulate the elongate tubing, and further allow for ease of insertion of the tubing within the patient, overcoming the shortcomings of the previous designs discussed in the background. The forceps device assists in allowing a practitioner in making smaller holes when inserting devices, such as elongate tubing, into a patient. Furthermore, the forceps device allows for fewer steps in the process of inserting a chest tube into a patient. The forceps device further decreases malpositioning of devices such as elongate tubing, and facilitates positioning of the tube direction within the patient. For example, having the tubing aligned with the channel of the clamping members allows for the tubing to be more accurately directed and positioned within the patient. This can allow for less trauma to a patient, and a faster healing process.

It is to be understood that the above description is intended to be illustrative, and not restrictive. Many other embodiments will be apparent to those of skill in the art upon reviewing the above description. The scope should, therefore, be determined with reference to the appended claims, along with the full scope of equivalents to which such claims are entitled.

1. A tube insertion device comprising:
a clamping portion operably coupled with a hinge portion, the clamping portion having opposing clamping members;
the clamping members extending from a first clamping member end to a second clamping member distal end;
the clamping members having a first open position and a second closed position;
the clamping members having opposing channels therein, the channel recessed away from a gripping surface of the clamping members; and
a handle portion operably coupled with the hinge portion.

2. The tube insertion device as recited in claim 1, wherein the channel is sized and configured to receive tubing therein without substantially collapsing a lumen of the tubing when the clamping members are disposed in the second closed position.

3. The tube insertion device as recited in claim 1, further comprising chest tubing disposed within the channel.

4. The tube insertion device as recited in claim 1, wherein at least a portion of the channels are aligned with longitudinal axes of the clamping members.

5. The tube insertion device as recited in claim 1, wherein the clamping portion is arcuately curved and has a radius.

6. The tube insertion device as recited in claim 1, wherein the clamping members include an intermediate portion between the first clamping member end and the second clamping member distal end, and at least one of the clamping members has an exit recess disposed along the intermediate portion.

7. The tube insertion device as recited in claim 1, wherein at least one channel has a U-shaped cross-section.

8. A tube insertion device comprising:
a handle portion operably coupled with a hinge portion, the hinge portion operable coupled with a clamping portion;
the clamping portion having opposing clamping members having a first open position and a second closed position;
the clamping members extending from a first clamping member end to a second clamping member distal end; and
at least one of the clamping members having a recess therein, the recess is sized and configured to receive tubing therein without substantially collapsing a lumen of the tubing when the clamping members are disposed in the second closed position.

9. The tube insertion device as recited in claim 8, wherein the clamping members include an intermediate portion between the first clamping member end and the second clamping member distal end, and at least one of the clamping members has an exit recess disposed along the intermediate portion.

10. The tube insertion device as recited in claim 8, further comprising a means for locking the clamping members in a closed position.

11. The tube insertion device as recited in claim 8, wherein the tube insertion device is at least one of a forceps, or a hemostat.

12. The tube insertion device as recited in claim 8, wherein at least a portion of the recess has an arcuate cross-section defining an inner surface.

13. The tube insertion device as recited in claim 12, further comprising elongate tubing disposed adjacent the inner surface of the recess.

14. The tube insertion device as recited in claim 8, wherein each clamping member has a recess, and the recesses of the clamping member oppose each other.

15. A method comprising:
opening and closing clamping members of a tube insertion device, the clamping members of the tube insertion device having a first outer diameter in a closed position;
disposing elongate tubing within a channel of at least one of the clamping members of the tube insertion device, the tubing having a lumen; and
clamping around a portion of elongate tubing without substantially collapsing the lumen of the tubing when the clamping members are disposed in the closed position.

16. The method as recited in claim 15, further comprising locking at least a portion of the tube insertion device when the clamping members are disposed in the closed position.

17. The method as recited in claim 15, further comprising disposing the elongate tubing through an exit recess disposed along an intermediate portion of the clamping members.

18. The method as recited in claim 15, wherein clamping the portion of elongate tubing includes clamping the elongate tubing without substantially increasing the first outer diameter when the clamping members are disposed in the closed position.

19. The method as recited in claim 15, further comprising placing a distal end of the elongate tubing adjacent to a channel distal end of the clamping member.

20. The method as recited in claim 15, wherein disposing elongate tubing within the channel includes disposing elongate tubing within at least two opposing channels of the clamping members.
